(19) United States
(12) Patent Application Publication
(10) Pub. No.: US 2025/0158118 A1
(43) Pub. Date: May 15, 2025

(54) SOLID ELECTROLYTE MEMBRANE, ELECTRODE, AND ALLSOLID- STATE RECHARGEABLE BATTERIES

(71) Applicant: SAMSUNG SDI CO., LTD., Yongin-si (KR)

(72) Inventors: Wonseok CHO, Yongin-si (KR); Inyeong Kang, Yongin-si (KR); Bongjin Moon, Goyang-si (KR)

(21) Appl. No.: 18/941,105

(22) Filed: Nov. 8, 2024

(30) Foreign Application Priority Data
Nov. 15, 2023 (KR) 10-2023-0158396

(51) Int. Cl. H01M 10/0565; C07D 491/08; H01M 10/052

(52) U.S. Cl. CPC H01M 10/0565; C07D 491/08; H01M 10/052; H01M 2300/0082

(57) ABSTRACT

A solid electrolyte membrane, an electrode including the solid electrolyte membrane, and an all-solid-state rechargeable battery including the solid electrolyte membrane, the solid electrolyte membrane includes a sulfide solid electrolyte; and an additive, wherein the additive includes a compound that is represented by Chemical Formula 1,

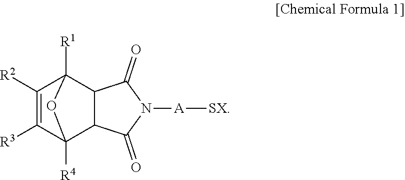

### CROSS-REFERENCE TO RELATED APPLICATION

This application claims priority to and the benefit of Korean Patent Application No. 10-2023-0158396 filed in the Korean Intellectual Property Office on Nov. 15, 2023, the entire contents of which are incorporated herein by reference.

### BACKGROUND

### 1. Field

Embodiments relate to a solid electrolyte membrane, an electrode, and an all-solid-state rechargeable battery.

### 2. Description of the Related Art

A portable information device such as a cell phone, a laptop, smart phone, or the like or an electric vehicle has used a rechargeable lithium battery having high energy density and easy portability as a driving power source. Recently, research has been actively conducted to use a rechargeable lithium battery with high energy density as a driving power source or power storage power source for hybrid or electric vehicles.

Some rechargeable lithium batteries may use electrolyte solutions including flammable organic solvents, and there may be safety issues such as explosion or fire of the batteries in the event of collision, penetration, or the like. Accordingly, an all-solid-state rechargeable battery using a solid electrolyte instead of an electrolyte solution has been considered. All-solid-state rechargeable batteries are batteries in which all materials are made of solid, and thus they may be safe as there is no risk of electrolyte solution leaking and exploding, and may have the advantage of being easy to manufacture thin batteries, and may reduce the thickness of the negative electrode, improving high-rate charging and discharging performance, and realizing high-voltage driving and high energy density.

### SUMMARY

The embodiments may be realized by providing a solid electrolyte membrane including a sulfide solid electrolyte; and an additive, wherein the additive includes a compound represented by Chemical Formula 1,

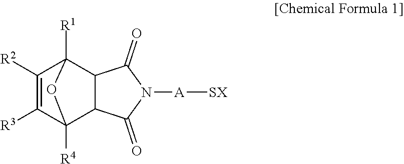

in Chemical Formula 1, A is a substituted or unsubstituted C1 to C20 alkylene group, a substituted or unsubstituted C3 to C20 cycloalkylene group, a substituted or unsubstituted C6 to C20 arylene group, a substituted or unsubstituted C6 to C20 heteroarylene group, or a combination thereof, S is sulfur, X is hydrogen, Li, Na, K, Rb, Cs, Fr, Mg, Ca, Ba, Cu, Zn, Ag, In, Sb, Co, Fe, Mn, or Pd, and R¹ to R⁴ are each independently hydrogen, a substituted or unsubstituted C1 to C20 alkyl group, a substituted or unsubstituted C2 to C20 alkenyl group, a substituted or unsubstituted C2 to C20 alkynyl group, a substituted or unsubstituted C6 to C20 aryl group, a substituted or unsubstituted C3 to C20 cycloalkyl group, a substituted or unsubstituted C3 to C20 cycloalkenyl group, a substituted or unsubstituted C3 to C20 cycloalkynyl group, a substituted or unsubstituted C2 to C20 heterocycloalkyl group, a substituted or unsubstituted C2 to C20 heterocycloalkenyl group, a substituted or unsubstituted C2 to C20 heterocycloalkynyl group, a substituted or unsubstituted C3 to C20 heteroaryl group, a substituted or unsubstituted C1 to C20 alkoxy group, an ester group, an ether group, a carboxyl group or a salt thereof, a cyano group, a carbonyl group, an imino group, a halogen, N, O, S, P, Si, B, or a combination thereof.

In Chemical Formula 1, A may be a substituted or unsubstituted C6 to C20 arylene group, a substituted or unsubstituted C6 to C20 heteroarylene group, or a combination thereof.

In Chemical Formula 1, X may be hydrogen, Li, Na, or K.

In Chemical Formula 1, R¹ to R⁴ may be each independently hydrogen, a substituted or unsubstituted C1 to C20 alkyl group, a substituted or unsubstituted C6 to C20 aryl group, a halogen, N, O, S, P, Si, B, or a combination thereof.

In Chemical Formula 1, A may be a substituted or unsubstituted C6 to C20 arylene group, X may be hydrogen, Li, or Na, and R¹ to R⁴ may be each independently hydrogen or a substituted or unsubstituted C1 to C20 alkyl group.

The additive may form a film in a temperature range of about 110° C. to about 180° C.

The additive may be included in an amount of about 1 wt % to about 10 wt %, based on a total weight of the solid electrolyte membrane.

The additive may be included in an amount of about 1 wt % to about 5 wt %, based on a total weight of the solid electrolyte membrane.

The solid electrolyte membrane may include a film including a compound represented by Chemical Formula 4:

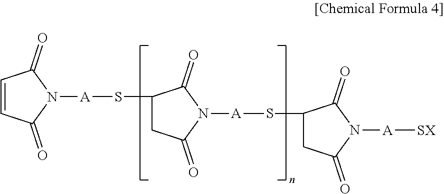

in Chemical Formula 4, A may be a substituted or unsubstituted C1 to C20 alkylene group, a substituted or unsubstituted C3 to C20 cycloalkylene group, a substituted or unsubstituted C6 to C20 arylene group, a substituted or unsubstituted C6 to C20 heteroarylene group, or a combination thereof, S may be sulfur, X may be hydrogen, Li, Na, K, Rb, Cs, Fr, Mg, Ca, Ba, Cu, Zn, Ag, In, Sb, Co, Fe, Mn, or Pd, and n may be an integer of 2 to 500.

In Chemical Formula 4, A may be a substituted or unsubstituted C6 to C20 arylene group, a substituted or unsubstituted C6 to C20 heteroarylene group, or a combination thereof.

In Chemical Formula 4, X may be hydrogen, Li, Na, or K.

In Chemical Formula 4, A may be a substituted or unsubstituted C6 to C20 arylene group, and X may be hydrogen, Li, or Na.

The sulfide solid electrolyte may be in the form of particles, and an average particle diameter (D50) of the particles may be about 0.1 μm to about 5.0 μm.

The sulfide solid electrolyte may include an argyrodite-type sulfide represented by Chemical Formula 5:

(LiₐM¹₆M²_{c})(P_{d}M³ₑ)(S_{f}M⁴_{g})Yₕ [Chemical Formula 5]
in Chemical Formula 5, 4≤a≤8, 0≤b<0.5, 0≤c<0.5, 0<d<4, 0≤e<1, 1.5≤n≤5, 3≤f≤12, 0≤g<2, and 0≤h≤2, M¹ may be Mg, Cu, Ag, or a combination thereof, M² may be Na, K, or a combination thereof, M³ may be Sn, Zn, Si, Sb, Ge, or a combination thereof, M⁴ may be O, SOₙ, or a combination thereof, and Y may be F, Cl, Br, I, or a combination thereof.

The embodiments may be realized by providing an electrode for an all-solid-state rechargeable battery, the electrode including n electrode active material, sulfide solid electrolyte particles, and an additive represented by Chemical Formula 1:

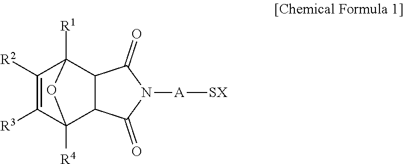

wherein, in Chemical Formula 1, A is a substituted or unsubstituted C1 to C20 alkylene group, a substituted or unsubstituted C3 to C20 cycloalkylene group, a substituted or unsubstituted C6 to C20 arylene group, a substituted or unsubstituted C6 to C20 heteroarylene group, or a combination thereof, S is sulfur, X is hydrogen, Li, Na, K, Rb, Cs, Fr, Mg, Ca, Ba, Cu, Zn, Ag, In, Sb, Co, Fe, Mn, or Pd, and R¹ to R⁴ are each independently hydrogen, a substituted or unsubstituted C1 to C20 alkyl group, a substituted or unsubstituted C2 to C20 alkenyl group, a substituted or unsubstituted C2 to C20 alkynyl group, a substituted or unsubstituted C6 to C20 aryl group, a substituted or unsubstituted C3 to C20 cycloalkyl group, a substituted or unsubstituted C3 to C20 cycloalkenyl group, a substituted or unsubstituted C3 to C20 cycloalkynyl group, a substituted or unsubstituted C2 to C20 heterocycloalkyl group, a substituted or unsubstituted C2 to C20 heterocycloalkenyl group, a substituted or unsubstituted C2 to C20 heterocycloalkynyl group, a substituted or unsubstituted C3 to C20 heteroaryl group, a substituted or unsubstituted C1 to C20 alkoxy group, an ester group, an ether group, a carboxyl group or a salt thereof, a cyano group, a carbonyl group, an imino group, a halogen, N, O, S, P, Si, B, or a combination thereof.

The additive may be included in an amount of about 1 wt % to about 10 wt %, based on a total weight of the electrode.

The embodiments may be realized by providing an all-solid-state rechargeable battery including a positive electrode, a negative electrode, and the solid electrolyte membrane according to an embodiment between the positive electrode and the negative electrode.

The negative electrode may include a current collector, a negative electrode coating layer on the current collector and including a lithiophilic metal, a carbon material, or a combination thereof, and a lithium metal layer between the current collector and the negative electrode coating layer, the lithium metal layer being formed by charging.

### BRIEF DESCRIPTION OF THE DRAWINGS

Features will be apparent to those of skill in the art by describing in detail exemplary embodiments with reference to the attached drawings in which:

FIGS. 1 and 2 are cross-sectional views schematically showing all-solid-state rechargeable batteries according to some embodiments.

FIG. 3 is a differential scanning calorimetry (DSC) graph for Reference Example 1.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In addition, it will also be understood that when a layer is referred to as being “between” two layers, it can be the only layer between the two layers, or one or more intervening layers may also be present. Like reference numerals refer to like elements throughout.

As used herein, “combination thereof” means a mixture, a laminate, a composite, a copolymer, an alloy, a blend, a reaction product, and the like of the constituents.

Herein, it should be understood that terms such as “comprises,” “includes,” or “have” are intended to designate the presence of an embodied feature, number, step, element, or a combination thereof, but it does not preclude the possibility of the presence or addition of one or more other features, number, step, element, or a combination thereof.

In the drawings, the thickness of layers, films, panels, regions, etc., are exaggerated for clarity and like reference numerals designate like elements throughout the specification. It will be understood that when an element such as a layer, film, region, or substrate is referred to as being “on” another element, it can be directly on the other element or intervening elements may also be present. In contrast, when an element is referred to as being “directly on” another element, there are no intervening elements present.

In addition, “layer” herein includes not only a shape formed on the whole surface when viewed from a plan view, but also a shape formed on a partial surface.

In addition, the average particle diameter may be measured by a method well known to those skilled in the art, for example, may be measured by a particle size analyzer, or may be measured by a transmission electron microscopic photograph or a scanning electron microscopic photograph. Alternatively, it is possible to obtain an average particle diameter value by measuring it using a dynamic light scattering method, performing data analysis, counting the number of particles for each particle size range, and calculating from this. Unless otherwise defined, the average particle diameter may mean the diameter (D₅₀) of particles having a cumulative volume of 50 volume % in the particle size distribution. As used herein, when a definition is not otherwise provided, the average particle diameter means a diameter (D₅₀) of particles having a cumulative volume of 50 volume % in the particle size distribution that is obtained by measuring the size (diameter or length of the major axis) of about 20 particles at random in a scanning electron microscope image.

Herein, “or” is not to be construed as an exclusive meaning, for example, “A or B” is construed to include A, B, A+B, and the like.

“Metal” is interpreted as a concept including ordinary metals, transition metals and metalloids (semi-metals).

As used herein, when specific definition is not otherwise provided, “substituted” refers to replacement of at least one hydrogen by a substituent of a halogen atom (F, Cl, Br, I), a hydroxy group, a C1 to C20 alkoxy group, a nitro group, a cyano group, an amine group, an imino group, an azido group, an amidino group, a hydrazino group, a hydrazono group, a carbonyl group, a carbamyl group, a thiol group, an ester group, an ether group, a carboxyl group or a salt thereof, sulfonic acid group or a salt thereof, a phosphoric acid group or a salt thereof, a C1 to C20 alkyl group, a C2 to C20 alkenyl group, a C2 to C20 alkynyl group, a C6 to C20 aryl group, a C3 to C20 cycloalkyl group, a C3 to C20 cycloalkenyl group, a C3 to C20 cycloalkynyl group, a C2 to C20 heterocycloalkyl group, a C2 to C20 heterocycloalkenyl group, a C2 to C20 heterocycloalkynyl group, a C3 to C20 heteroaryl group, or a combination thereof.

### Solid Electrolyte Membrane

According to an embodiment, a solid electrolyte membrane may include, e.g., a sulfide solid electrolyte and an additive. In an implementation, the additive may include a compound represented by Chemical Formula 1, below.

### Additive

The additive may form a film through a polymerization reaction or the like, e.g., in response to a rise in temperature due to Joule heat situations, e.g., penetration, a short circuit, or the like of a battery, to thereby block an ion current and thus induce a shutdown.

The additive may begin a reaction for forming the film, e.g., at greater than or equal to about 110° C. or greater than or equal to about 120° C., and thus may form the film at a temperature lower than about 180° C., which is a melting point of lithium metal, to thereby help effectively suppress desorption or permeation of liquid lithium.

In an implementation, the additive may form the film at about 110° C. to about 180° C., e.g., about 120° C. to about 180° C., about 140° C. to about 170° C., or about 160° C. to about 170° C. The additive may be expressed or referred to as a film-forming additive, a shutdown additive, or the like.

The additive may have no or almost no reactivity with a sulfide solid electrolyte in the solid electrolyte membrane, and thus may not deteriorate the solid electrolyte. In an implementation, the film may be formed even in a small amount of the additive, and ionic conductivity of the solid electrolyte membrane may not be deteriorated, but high-temperature safety may be improved without deteriorating performance. The additive may be dispersed in the solid electrolyte membrane and thus may act as a type of dispersant or have no influence on the solid electrolyte membrane during normal reaction or battery operation. If the battery were to be subjected to high temperature conditions, e.g., due to an abnormal reaction or situation, the film may be formed to perform a shutdown function.

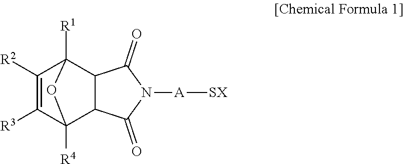

In Chemical Formula 1, A may be or may include, e.g., a substituted or unsubstituted C1 to C20 alkylene group, a substituted or unsubstituted C3 to C20 cycloalkylene group, a substituted or unsubstituted C6 to C20 arylene group, a substituted or unsubstituted C6 to C20 heteroarylene group, or a combination thereof.

S is sulfur.

X may be hydrogen, Li, Na, K, Rb, Cs, Fr, Mg, Ca, Ba, Cu, Zn, Ag, In, Sb, Co, Fe, Mn, or Pd.

R¹ to R⁴ may each independently be or include, e.g., hydrogen, a substituted or unsubstituted C1 to C20 alkyl group, a substituted or unsubstituted C2 to C20 alkenyl group, a substituted or unsubstituted C2 to C20 alkynyl group, a substituted or unsubstituted C6 to C20 aryl group, a substituted or unsubstituted C3 to C20 cycloalkyl group, a substituted or unsubstituted C3 to C20 cycloalkenyl group, a substituted or unsubstituted C3 to C20 cycloalkynyl group, a substituted or unsubstituted C2 to C20 heterocycloalkyl group, a substituted or unsubstituted C2 to C20 heterocycloalkenyl group, a substituted or unsubstituted C2 to C20 heterocycloalkynyl group, a substituted or unsubstituted C3 to C20 heteroaryl group, a substituted or unsubstituted C1 to C20 alkoxy group, an ester group, an ether group, a carboxyl group or a salt thereof, a cyano group, a carbonyl group, an imino group, a halogen, N, O, S, P, Si, B, or a combination thereof. In an implementation, at least one of R¹ to R⁴ may include one of the multivalent groups or atoms described above, e.g., an ester group, an ether group, N, O, or the like, in combination with (e.g., capped by) at least one of the monovalent groups described above (e.g., R¹ could include a combination of N and hydrogen to be a —NH₂ group).

In an implementation, A may be a substituted or unsubstituted C6 to C20 arylene group, a substituted or unsubstituted C6 to C20 heteroarylene group, a combination thereof, e.g., a substituted or unsubstituted C6 to C20 arylene group. In an implementation, A may be a substituted or unsubstituted phenylene group or a substituted or unsubstituted naphthalene group.

In an implementation, X may be hydrogen, Li, Na, K, Rb, Cs, or Fr, e.g., hydrogen, Li, Na, or K.

In an implementation, the compound represented by Chemical Formula 1 may be lithiated or sodiumized and thus may contain lithium ions or sodium ions. In Chemical Formula 1, X may be, e.g., hydrogen, lithium, or sodium. In an implementation, a portion of the X in Chemical Formula 1 may be hydrogen and the remaining portion may be lithium or sodium. In an implementation, the compound represented by Chemical Formula 1 and a lithium salt may be mixed in the solid electrolyte and the compound represented by Chemical Formula 1 and a sodium salt may be mixed. The lithiated or sodiumized additives may help further improve the ionic conductivity of the solid electrolyte membrane.

In an implementation, R¹ to R⁴ may each independently be, e.g., hydrogen, a substituted or unsubstituted C1 to C20 alkyl group, a substituted or unsubstituted C6 to C20 aryl group, a halogen, N, O, S, P, Si, B, combination thereof, e.g., hydrogen or a substituted or unsubstituted C1 to C20 alkyl group.

In an implementation, A may be, e.g., a substituted or unsubstituted C6 to C20 arylene group, X may be, e.g., hydrogen, Li, or Na, and R¹ to R⁴ may each independently be, e.g., hydrogen or a substituted or unsubstituted C1 to C20 alkyl group.

In an implementation, the additive may form a film in a temperature range of about 110° C. to about 180° C., e.g., about 120° C. to about 180° C., about 130° C. to about 180° C., about 140° C. to about 180° C., or about 150° C. to about 170° C.

The compound represented by Chemical Formula 1 may undergo a retro-Diels-Alder reaction, which is a thermal decomposition reaction, at high temperature conditions of about 110° C. to about 180° C., and may be decomposed into a conjugated diene compound represented by Chemical Formula 2 and an maleimide alkene compound represented by Chemical Formula 3.

In an implementation, the thermal decomposition temperature may vary depending on the type of A in Chemical Formula 1.

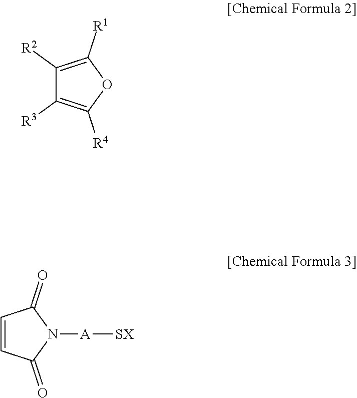

The maleimide compound represented by Chemical Formula 3 may be an unstable compound in which a maleimide moiety with positive (+) charge and an SX moiety with a negative (−) charge exist. The maleimide moiety and the SX moiety may undergo a continuous click reaction to form a polymer or compound, and such a polymer or compound may form a film on the solid electrolyte membrane.

In an implementation, at high temperatures, the maleimide compound represented by Chemical Formula 3 may react with —SH on the surface of the argyrodite-type sulfide solid electrolyte, drastically reducing the ionic conductivity of the solid electrolyte and inducing a shutdown, thereby improving the safety of the battery.

In an implementation, the solid electrolyte membrane according to some embodiments may include a film including a polymer or compound represented by Chemical Formula 4.

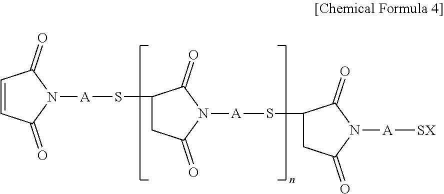

In Chemical Formula 4, A may be or may include, e.g., a substituted or unsubstituted C1 to C20 alkylene group, a substituted or unsubstituted C3 to C20 cycloalkylene group, a substituted or unsubstituted C6 to C20 arylene group, a substituted or unsubstituted C6 to C20 heteroarylene group, or a combination thereof.

S is sulfur. X may be, e.g., hydrogen, Li, Na, K, Rb, Cs, Fr, Mg, Ca, Ba, Cu, Zn, Ag, In, Sb, Co, Fe, Mn, or Pd.

n may be an integer of, e.g., 2 to 500.

In an implementation, A may be, e.g., a substituted or unsubstituted C6 to C20 arylene group, a substituted or unsubstituted C6 to C20 heteroarylene group, a combination thereof. In an implementation, A may be, e.g., a substituted or unsubstituted C6 to C20 arylene group.

In an implementation, X may be hydrogen, Li, Na, K, Rb, Cs, or Fr, e.g., hydrogen, Li, or Na.

In an implementation, A may be, e.g., a substituted or unsubstituted C6 to C20 arylene group and X may be, e.g., hydrogen, Li, Na, or K.

In an implementation, in Chemical Formula 4, n refers to the number of repetitions of the repeating or structural unit in the polymer (or oligomer or compound), and may be, e.g., an integer of 2 to 500, an integer of 3 to 400, an integer of 4 to 300, an integer of 5 to 200, an integer from 10 to 100, an integer from 10 to 50, or an integer from 2 to 20.

In an implementation, the additive may be included in an amount of about 1 wt % to about 10 wt %, about 1 wt % to about 5 wt %, or about 3 wt % to about 5 wt %, based on a total weight of the solid electrolyte membrane. Within the above content ranges, a film may be effectively formed under high temperature conditions without impairing the performance of the solid electrolyte membrane.

### Sulfide Solid Electrolyte

A sulfide solid electrolyte may include, e.g., Li₂S—P₂S₅, Li₂S—P₂S₅—LiY (wherein Y is a halogen element, for example I or Cl), Li₂S—P₂S₅—Li₂O, Li₂S—P₂S₅—Li₂O—LiI, Li₂S—SiS₂, Li₂S—SiS₂—LiI, Li₂S—SiS₂—LiBr, Li₂S—SiS₂—LiCl, Li₂S—SiS₂—B₂S₃—LiI, Li₂S—SiS₂—P₂S₅—LiI, Li₂S—B₂S₃, Li₂S—P₂S₅—ZₘSₙ (wherein m and n is each an integer and Z is Ge, Zn, or Ga), Li₂S—GeS₂, Li₂S—SiS₂—Li₃PO₄, Li₂S—SiS₂-LiₚMO_{q} (wherein p and q each an integer and Mis P, Si, Ge, B, Al, Ga, or In), or a combination thereof.

Such a sulfide solid electrolyte may be obtained by, e.g., mixing Li₂S and P₂S₅ in a mole ratio of about 50:50 to about 90:10 or about 50:50 to about 80:20 and optionally, performing heat treatment. Within the above mixing ratio ranges, a sulfide solid electrolyte having excellent ionic conductivity may be prepared. The ionic conductivity may be further improved by adding SiS₂, GeS₂, B₂S₃, or the like as other components thereto.

Mechanical milling or a solution method may be applied as a mixing method of sulfur-containing raw materials for preparing a sulfide solid electrolyte. The mechanical milling may be to make starting materials into particulates by putting the starting materials like in a ball mill reactor and vigorously stirring them. The solution method may be performed by mixing the starting materials in a solvent to obtain a solid electrolyte as a precipitate. In addition, in the case of heat treatment after mixing, crystals of the solid electrolyte may be more robust and ionic conductivity may be improved. In an implementation, the sulfide solid electrolyte may be prepared by mixing sulfur-containing raw materials and performing heat treatment two or more times. In this case, a sulfide solid electrolyte having high ionic conductivity and robustness may be prepared.

The sulfide solid electrolyte particles according to some example embodiments, e.g., may be prepared through a first heat treatment of mixing sulfur-containing raw materials and firing at about 120° C. to about 350° C. and a second heat treatment of mixing the resultant of the first heat treatment and firing the same at about 350° C. to about 800° C. The first heat treatment and the second heat treatment may be performed under an inert gas or nitrogen atmosphere, respectively. The first heat treatment may be performed for about 1 hour to about 10 hours, and the second heat treatment may be performed for about 5 hours to about 20 hours. Small raw materials may be milled through the first heat treatment, and a final solid electrolyte may be synthesized through the second heat treatment. Through such two or more heat treatments, a sulfide solid electrolyte having high ionic conductivity and high performance may be obtained, and such a solid electrolyte may be suitable for mass production. The temperature of the first heat treatment may be, e.g., about 150° C. to about 330° C., or about 200° C. to about 300° C., and the temperature of the second heat treatment may be, e.g., about 380° C. to about 700° C., or about 400° C. to about 600° C.

In an implementation, the sulfide solid electrolyte particles may include argyrodite-type sulfide. The argyrodite-type sulfide solid electrolyte may have high ionic conductivity close to the range of about 10-4 to about 10-2 S/cm, which is the ionic conductivity of some liquid electrolytes at room temperature, and may form an intimate bond between the positive electrode active material and the solid electrolyte without causing a decrease in ionic conductivity, and furthermore, an intimate interface between the electrode layer and the solid electrolyte layer. An all-solid-state battery including the same may have improved battery performance such as rate capability, coulombic efficiency, and cycle-life characteristics.

In an implementation, the argyrodite-type sulfide solid electrolyte particles may include a compound represented by Chemical Formula 5.

(LiₐM¹_{b}M²_{c})(P_{d}M³ₑ)(S_{f}M⁴_{g})Yₕ [Chemical Formula 5]

In Chemical Formula 5, 4≤a≤8, M¹ may be Mg, Cu, Ag, or a combination thereof, 0≤b<0.5, M² may be Na, K, or a combination thereof, 0≤c<0.5, M³ may be Sn, Zn, Si, Sb, Ge, or a combination thereof, 0<d<4, 0≤e<1, M⁴ may be O, SOₙ, or a combination thereof, 1.5≤n≤5, 3≤f≤12, 0≤g<2, Y may be F, Cl, Br, I, or a combination thereof, and 0≤h≤2.

In an implementation, the halide element (Y) may be necessarily included in Chemical Formula 5, and in this case, it may be expressed as 0<h≤2. In an implementation, M¹ element may be necessarily included in Chemical Formula 5, and in this case, it may be expressed as 0<b<0.5. In Chemical Formula 5, M³ may be understood as an element substituted for P and may be 0<e<1. In Chemical Formula 5, M⁴ may be substituted for S, e.g., may be 0<g<2, and f, a ratio of S, may be, e.g., 3≤f≤7. In an implementation, M⁴ may be SOₙ, and SOₙ may be, e.g., S₄O₆, S₃O₆, S₂O₃, S₂O₄, S₂O₅, S₂O₆, S₂O₇, S₂O₈, SO₄, SO₅, or the like.

In an implementation, in Chemical Formula 5, a+b+c+h=7, d+e=1, and f+g+h=6.

In an implementation, the argyrodite-type sulfide solid electrolyte particles may include, e.g., Li₃PS₄, Li₂P₃S₁₁, Li₇PS₆, Li₆PS₅Cl, Li₆PS₅Br, Li_{5.8}PS_{4.8}Cl_{1.2}, Li_{6.2}PS_{5.2}Br_{0.8}, Li_{5.75}PS_{4.75}Cl_{1.25}, (Li_{5.69}Cu_{0.06})PS_{4.75}Cl_{1.25}, (Li_{5.72}Cu_{0.03}) PS_{4.75}Cl_{1.25}, (Li_{5.69}Cu_{0.06})P(S_{4.70}(SO₄)_{0.05})Cl_{1.25}, (Li_{5.69}Cu_{0.06})P(S_{4.60}(SO₄)_{0.15})Cl_{1.25}, (Li_{5.72}Cu_{0.03})P(S_{4.725}(SO₄)_{0.025})Cl_{1.25}, (Li_{5.72}Na_{0.03})P(S_{4.725}(SO₄)_{0.025})Cl_{1.25}, Li_{5.75}P(S_{4.725}(SO₄)_{0.025})Cl_{1.25}, or a combination thereof.

The argyrodite-type sulfide solid electrolyte may be prepared, e.g., by mixing lithium sulfide and phosphorus sulfide, and optionally lithium halide. Heat treatment may be performed after mixing them. The heat treatment may include, e.g., two or more heat treatment steps. Herein, the of preparing the argyrodite-type sulfide solid electrolyte may include, e.g., a first heat treatment in which raw materials are mixed and fired at about 120° C. to about 350° C., and a second heat treatment in which the resultant of the first heat treatment is mixed again and fired at about 350° C. to about 800° C.

An average particle diameter (D₅₀) of the sulfide solid electrolyte particles may be for example small particles of about 0.1 μm to about 5.0 μm or about 0.1 μm to about 3.0 μm, or about 0.1 μm to about 1.9 μm or large particles of about 2.0 μm to about 5.0 μm. The sulfide solid electrolyte particles may be a mixture of small particles with an average particle diameter of about 0.1 μm to about 1.9 μm and large particles with an average particle diameter of about 2.0 μm to about 5.0 μm. The average particle diameter of the sulfide solid electrolyte particles may be measured using an electron microscope image, and for example, a particle size distribution may be obtained by measuring the size (diameter or length of the major axis) of about 20 particles in a scanning electron microscope image, and D₅₀ may be calculated therefrom.

### Binder

The solid electrolyte membrane according to some embodiments may further include a binder. In an implementation, the binder may include a nitrile-butadiene rubber, hydrogenated nitrile-butadiene rubber, a styrene-butadiene rubber, an acrylated styrene-butadiene rubber, an acrylonitrile-butadiene rubber, an acrylic rubber, a butyl rubber, a fluorine rubber, a natural rubber, polydimethylsiloxane, polyethyleneoxide, polyvinylpyrrolidone, polyvinylpyridine, chlorosulfonated polyethylene, polyvinyl alcohol, polytetrafluoroethylene, polyvinylidene fluoride, a polyvinylidene fluoride-hexafluoropropylene copolymer, polyvinylchloride, carboxylated polyvinylchloride, polyvinylfluoride, polyethylene, polypropylene, an ethylene-propylene copolymer, an ethylene-propylene-diene copolymer, polyamideimide, polyimide, poly(meth)acrylate, polyacrylonitrile, polystyrene, polyurethane, a copolymer thereof, or a combination thereof.

The binder may be included in an amount of about 0.1 wt % to about 3 wt %, e.g., about 0.5 wt % to about 2 wt %, or about 0.5 wt % to about 1.5 wt %, based on a total weight of the solid electrolyte membrane. Including the binder within the above ranges may help ensure that the components in the solid electrolyte membrane can be well combined without reducing the ionic conductivity of the solid electrolyte, thereby improving the durability and reliability of the battery.

### Other Components

In an implementation, the solid electrolyte membrane may include an oxide inorganic solid electrolyte in addition to the sulfide solid electrolyte. In an implementation, the oxide inorganic solid electrolyte may include, e.g., Li₁₊ₓTi₂₋ₓAl(PO₄)₃(LTAP) (0≤x≤4), Li_{1+x+y}AlₓTi₂₋ₓSi_{y}P_{3-y}O₁₂ (0<x<2, 0≤y<3), BaTiO₃, Pb(Zr,Ti)O₃(PZT), Pb₁₋ₓLaₓZr_{1-y}Ti_{y}O₃ (PLZT) (0≤x<1, 0≤y<1), PB(Mg₃Nb_{2/3})O₃—PbTiO₃ (PMN-PT), HfO₂, SrTiO₃, SnO₂, CeO₂, Na₂O, MgO, NiO, CaO, BaO, ZnO, ZrO₂, Y₂O₃, Al₂O₃, TiO₂, SiO₂, lithium phosphate (Li₃PO₄), lithium titanium phosphate (LiₓTi_{y}(PO₄)₃, 0<x<2, 0<y<3), Li_{1+x+y}(Al, Ga)ₓ(Ti, Ge)₂₋ₓSi_{y}P_{3-y}O₁₂ (0≤x≤1, 0≤y≤1), lithium lanthanum titanate (LiₓLa_{y}TiO₃, 0<x<2, 0<y<3), Li₂O, LiAlO₂, Li₂O—Al₂O₃—SiO₂—P₂O₅—TiO₂—GeO₂ ceramics, Garnet ceramics Li₃₊ₓLa₃M₂O₁₂ (wherein M=Te, Nb, or Zr; x is an integer of 1 to 10), or a mixture thereof.

In an implementation, the solid electrolyte membrane may further include a halide solid electrolyte. The halide solid electrolyte may include a halogen element as a main component, meaning that a ratio of the halide element to all elements of the solid electrolyte may be greater than or equal to about 50 mol %, greater than or equal to about 70 mol %, greater than or equal to about 90 mol %, or 100 mol %. In an implementation, the halide solid electrolyte may not contain a sulfur element.

In an implementation, the halide solid electrolyte may contain a lithium element, a metal element other than lithium, and a halogen element. The metal element other than lithium may include Al, As, B, Bi, Ca, Cd, Co, Cr, Fe, Ga, Hf, In, Mg, Mn, Ni, Sb, Sc, Sn, Ta, Ti, Y, Zn, Zr, or a combination thereof. The halogen element may be F, Cl, Br, I, or a combination thereof, e.g., C1, Br, or a combination thereof. In an implementation, the halide solid electrolyte may be, e.g., LiₐM₁X₆ (M is Al, As, B, Bi, Ca, Cd, Co, Cr, Fe, Ga, Hf, In, Mg, Mn, Ni, Sb, Sc, Sn, Ta, Ti, Y, Zn, Zr, or a combination thereof, X is F, Cl, Br, I, or a combination thereof, and 2≤a≤3). In an implementation, the halide solid electrolyte may include, e.g., Li₂ZrCl₆, Li_{2.7}Y_{0.7}Zr_{0.3}Cl₆, Li_{2.5}Y_{0.5}Zr_{0.5}Cl₆, Li_{2.5}In_{0.5}Zr_{0.5}Cl₆, Li₂In_{0.5}Zr_{0.5}Cl₆, Li₃YBr₆, Li₃YCl₆, Li₃YBr₂Cl₄, Li₃YbCl₆, Li_{2.6}Hf_{0.4}Yb_{0.6}Cl₆, or a combination thereof.

In an implementation, the solid electrolyte membrane may further include an alkali metal salt, an ionic liquid, or a conductive polymer.

In an implementation, the alkali metal salt may be lithium salt. The concentration of lithium salt in the solid electrolyte layer may be greater than or equal to about 1 M, e.g., about 1 M to about 4 M. In this case, the lithium salt may help improve ionic conductivity by improving lithium ion mobility in the solid electrolyte layer.

The lithium salt may include, e.g., LiPF₆, LiBF₄, LiSbF₆, LiAsF₆, LiClO₄, LiAlO₂, LiAlCl₄, LiPO₂F₂, LiCl, LiI, LiSCN, LiN(CN)₂, lithium bis(oxalato)borate (LiBOB), lithium difluorobis(oxalato)borate (LiDFOB), lithium difluorobis(oxalato)phosphate (LiDFBP), lithium bis(trifluoromethanesulfonyl)imide (LiTFSI), lithium bis(fluorosulfonyl)imide (LiFSI), lithium bis(pentafluoroethanesulfonyl)imide (LiBETI), lithium trifluoromethane sulfonate, lithium tetrafluoroethane sulfonate, or a combination thereof.

In an implementation, the lithium salt may be an imide lithium salt, e.g., LiTFSI, LiFSI, LiBETI, or a combination thereof. The imide lithium salt may help maintain or improve ionic conductivity by maintaining appropriate chemical reactivity with ionic liquids.

The ionic liquid has a melting point below room temperature, so it is in a liquid state at room temperature and refers to a salt or room temperature molten salt composed of ions alone.

The ionic liquid may be a compound including a cation, e.g., ammonium, pyrrolidinium, pyridinium, pyrimidinium, imidazolium, piperidinium, pyrazolium, oxazolium, pyridazinium, phosphonium, sulfonium, triazolium, or a mixture thereof, and an anion, e.g., BF₄⁻, PF₆⁻, AsF₆⁻, SbF₆⁻, AlCl₄⁻, HSO₄⁻, ClO₄⁻, CH₃SO₃⁻, CF₃CO₂⁻, Cl⁻, Br⁻, I⁻, BF₄⁻, SO₄⁻, CF₃SO₃—, (FSO₂)₂N⁻, (C₂F₅SO₂)2N⁻, (C₂F₅SO₂)(CF₃SO₂)N⁻, or (CF₃SO₂)₂N⁻.

The ionic liquid may include, e.g., N-methyl-N-propylpyrrolidinium bis(trifluoromethanesulfonyl)imide N-butyl-N-methylpyrrolidium bis(3-trifluoromethylsulfonyl)imide, 1-butyl-3-methylimidazolium bis(trifluoromethylsulfonyl)amide, or 1-ethyl-3-methylimidazolium bis(trifluoromethylsulfonyl)amide.

A weight ratio of the solid electrolyte and the ionic liquid in the solid electrolyte layer may be about 0.1:99.9 to about 90:10, e.g., about 10:90 to about 90:10, about 20:80 to about 90:10, about 30:70 to about 90:10, about 40:60 to about 90:10, or about 50:50 to about 90:10. The solid electrolyte layer satisfying the above ranges may maintain or improve ionic conductivity by improving the electrochemical contact area with the electrode. Accordingly, the energy density, discharge capacity, rate capability, etc. of the all-solid-state rechargeable battery may be improved.

Some embodiments provide an electrode for an all-solid-state rechargeable battery including an electrode active material, solid electrolyte particles, and the aforementioned additive represented by Chemical Formula 1.

In an implementation, the aforementioned additive may be included in the electrode in addition to the solid electrolyte membrane, and if the temperature were to rise due to an issue that generates heat at a high temperature, a film may be formed on the electrode through a polymerization reaction of the additive, or the additive and the solid electrolyte particles may react to increase the ionic conductivity of the solid electrolyte, and by rapidly decreasing the voltage and inducing a shutdown, the safety of the battery can be improved.

The additive may be included in an amount of about 1 wt % to about 10 wt %, e.g., about 1 wt % to about 5 wt %, or about 3 wt % to about 5 wt %, based on a total weight of the electrode. Within the above content ranges, a film may be effectively formed under high temperature conditions without impairing electrode performance.

In an implementation, the electrode may be a positive electrode, and in some embodiments, a positive electrode for an all-solid-state rechargeable battery including a positive electrode active material, solid electrolyte particles, and the additive represented by Chemical Formula 1 may be provided. In an implementation, the electrode may be a negative electrode, and accordingly, a negative electrode for an all-solid-state rechargeable battery including a negative electrode active material, solid electrolyte particles, and the additive represented by Chemical Formula 1 may be provided.

The additive may be the same as described above.

### Positive Electrode

In an implementation, the positive electrode may include a current collector and a positive electrode active material layer on the current collector. In an implementation, the positive active material layer may include a positive electrode active material and a solid electrolyte and may optionally include a binder and/or a conductive material, and may include optionally the aforementioned additive represented by Chemical Formula 1. In an implementation, the positive electrode active material layer may include the aforementioned solid electrolyte.

### Positive Electrode Active Material

The positive electrode active material may be a suitable material used in all-solid-state rechargeable batteries. In an implementation, the positive electrode active material may be a compound being capable of intercalating and deintercalating lithium, may include a lithium transition metal composite oxide, and may include a compound represented by any of the following chemical formulas.

LiₐA_{1-b}X_{b}D′₂(0.90≤a≤1.8,0≤b≤0.5);

LiₐA_{1-b}X_{b}O_{2-c}D′_{c}(0.90≤a≤1.8,0≤b≤0.5,0≤c≤0.05);

LiₐE_{1-b}X_{b}O_{2-c}D′_{c}(0.90≤a≤1.8,0≤b≤0.5,0≤c≤0.05);

LiₐE_{2-b}X_{b}O_{4-c}D′_{c}(0.90≤a≤1.8,0≤b≤0.5,0≤c≤0.05);

LiₐNi_{1-b-c}CO_{b}X_{c}D′_{α}(0.90≤a≤1.8,0≤b≤0.5,0≤c≤0.5,0<α≤2);

LiₐNi_{1-b-c}Co_{b}X_{c}O_{2-α}T_{α}(0.90≤a≤1.8,0≤b≥0.5,0≤c≤0.05,0<α≤2);

LiₐNi_{1-b-c}CO_{b}X_{c}O_{2-α}T₂(0.90≤a≤1.8,0≤b≤0.5,0≤c≤0.05,0<α<2);

LiₐNi_{1-b-c}Mn_{b}X_{c}D_{α}(0.90≤a≤1.8,0≤b≤0.5,0≤c≤0.05,0<α≤2);

LiₐNi_{1-b-c}Mn_{b}X_{c}O_{2-α}T_{α}(0.90≤a≤1.8,0≤b≤0.5,0≤c≤0.05,0<α<2);

LiₐNi_{1-b-c}Mn_{b}X_{c}O_{2-α}T₂(0.90≤a≤1.8,0≤b≤0.5,0≤c≤0.05,0<α<2);

LiₐNi_{b}E_{c}G_{d}O₂(0.90≤a≤1.8,0≤b≤0.9,0≤c≤0.5,0.001≤d≤0.1);

LiₐNi_{b}Co_{c}Mn_{d}GₑO₂(0.90≤a≤1.8,0≤b≤0.9,0≤c≤0.5,0≤d<0.5,0.001≤e≤0.1);

LiₐNiG_{b}O₂(0.90≤a≤1.8,0.001≤b≤0.1);

LiₐCoG_{b}O₂(0.90≤a≤1.8,0.001≤b≤0.1);

LiₐMn_{1-b}G_{b}O₂(0.90≤a≤1.8,0.001≤b≤0.1);

LiₐMn₂G_{b}O₄(0.90≤a≤1.8,0.001≤b≤0.1);

LiₐMn_{1-g}G_{g}PO₄(0.90≤a≤1.8,0≤g≤0.5);

QO₂;QS₂;LiQS₂;

V₂O₅;LiV₂O₅;

LiZO₂;

LiNiVO₄;

Li_{(3-f)}J₂(PO₄)₃(0≤f≤2);

Li_{(3-f)}Fe₂(PO₄)₃(0≤f≤2); and

LiₐFePO₄(0.90≤a≤1.8).

In the chemical formulas, A may be Ni, Co, Mn, or a combination thereof; X may be Al, Ni, Co, Mn, Cr, Fe, Mg, Sr, V, a rare earth element, or a combination thereof; D′ may be O, F, S, P, or a combination thereof; E may be Co, Mn, or a combination thereof; T may be F, S, P, or a combination thereof; G may be Al, Cr, Mn, Fe, Mg, La, Ce, Sr, V, or a combination thereof; Q may be Ti, Mo, Mn, or a combination thereof; Z may be Cr, V, Fe, Sc, Y, or a combination thereof; and J may be V, Cr, Mn, Co, Ni, Cu, or a combination thereof.

The positive electrode active material may be, e.g., a lithium cobalt oxide (LCO), a lithium nickel oxide (LNO), a lithium nickel cobalt oxide (NC), a lithium nickel cobalt aluminum oxide (NCA), a lithium nickel cobalt manganese oxide (NCM), a lithium nickel manganese oxide (NM), a lithium manganese oxide (LMO), or lithium iron phosphate (LFP).

In an implementation, the positive electrode active material may include a lithium nickel oxide represented by Chemical Formula 6, a lithium cobalt oxide represented by Chemical Formula 7, a lithium iron phosphate compound represented by Chemical Formula 8, and cobalt-free lithium nickel-manganese oxide represented by Chemical Formula 9, or a combination thereof.

Liₐ₁Niₓ₁M¹_{y1}M²_{z1}O_{2-b1}X_{b1} [Chemical Formula 6]

In Chemical Formula 6, 0.9≤a1≤1.8, 0.3≤x1≤1, 0≤y1≤0.7, 0≤z1≤0.7, 0.9≤x1+y1+z1≤1.1, 0≤b1≤0.1, M¹ and M² may each independently be Al, B, Ba, Ca, Ce, Co, Cr, Cu, Fe, Mg, Mn, Mo, Nb, Si, Sn, Sr, Ti, V, W, or Zr, and X may be F, P, or S.

In Chemical Formula 1, 0.6≤x1≤1, 0≤y1≤0.4, and 0≤z1≤0.4 or 0.8≤x1≤1, 0≤y1≤0.2, and 0≤z1≤0.2.

Liₐ₂Coₓ₂M³_{y2}O_{2-b2}X_{b2} [Chemical Formula 7]

In Chemical Formula 7, 0.9≤a2≤1.8, 0.7≤x2≤1, 0≤y2≤0.3, 0.9≤x2+y2≤1.1, and 0≤b2≤0.1, M³ may be Al, B, Ba, Ca, Ce, Cr, Cu, Fe, Mg, Mn, Mo, Ni, Se, Si, Sn, Sr, Ti, V, W, Y, Zn, or Zr, and X may be F, P, or S.

Liₐ₃Feₓ₃M⁴_{y3}PO_{4-b3}X_{b3} [Chemical Formula 8]

In Chemical Formula 8, 0.9≤a3≤1.8, 0.6≤x3≤1, 0≤y3≤0.4, 0<b3≤0.1, M⁴ may be Al, B, Ba, Ca, Ce, Co, Cr, Cu, Mg, Mn, Mo, Ni, Se, Si, Sn, Sr, Ti, V, W, Y, Zn, or Zr, and X may be F, P, or S.

Liₐ₄Niₓ₄Mn_{y4}M⁵_{z4}O_{2-b4}X_{b4} [Chemical Formula 9]

In Chemical Formula 9, 0.9≤a2≤1.8, 0.8≤x4<1, 0<y4<0.2, 0≤z4≤0.2, 0.9≤x4+y4+z4<1.1, 0_b4<0.1, M⁵ may be Al, B, Ba, Ca, Ce, Cr, Fe, Mg, Mo, Nb, Si, Sn, Sr, Ti, V, W, or Zr, and X may be F, P, or S.

An average particle diameter (D₅₀) of the positive electrode active material may be about 1 μm to about 25 μm, e.g. about 3 μm to about 25 μm, about 1 μm to about 20 μm, about 1 μm to about 18 μm, about 3 μm to about 15 μm, or about 5 μm to about 15 μm. In an implementation, the positive electrode active material may include small particles having an average particle diameter (D₅₀) of about 1 μm to about 9 μm and large particles having an average particle diameter (D₅₀) of about 10 μm to about 25 μm. The positive electrode active material having this particle size range can be harmoniously mixed with other components within the positive active material layer and can achieve high capacity and high energy density. Herein, the average particle diameter means a diameter (D₅₀) of particles having a cumulative volume of 50 volume % in the particle size distribution that is obtained by measuring the size (diameter or length of the major axis) of about 20 particles at random in a scanning electron microscope image for positive electrode active materials.

The positive electrode active material may be in the form of secondary particles made by agglomerating a plurality of primary particles or in the form of single particles. In an implementation, the positive electrode active material may have a spherical or close to spherical shape, or may have a polyhedral or irregular shape.

In an implementation, the positive electrode active material may include a buffer layer on the surface of the particles. The buffer layer may be expressed or referred to as a coating layer, a protective layer, etc., and may help lower the interfacial resistance between the positive electrode active material and the sulfide solid electrolyte particles. In an implementation, the buffer layer may include lithium-metal-oxide, and the metal may include Al, B, Ca, Ce, Cr, Fe, Mg, Mo, Nb, Si, Sn, Sr, Ta, V, W, or Zr. The lithium-metal-oxide may help improve the performance of the positive electrode active material by facilitating the movement of lithium ions and electronic conduction, and is improved for lowering the interfacial resistance between the positive electrode active material and solid electrolyte particles.

The positive electrode active material may be included in an amount of about 55 wt % to about 99 wt %, e.g., about 65 wt % to about 95 wt %, or about 75 wt % to about 91 wt %, based on a total weight of the positive electrode active material layer.

### Binder

The binder may adhere the positive electrode active material particles to each other and also may properly attach the positive active material to the current collector. Examples thereof may include polyvinyl alcohol, carboxylmethyl cellulose, hydroxypropyl cellulose, diacetyl cellulose, polyvinylchloride, carboxylated polyvinylchloride, polyvinylfluoride, an ethylene oxide-containing polymer, polyvinylpyrrolidone, polyurethane, polytetrafluoroethylene, polyvinylidene fluoride, polyethylene, polypropylene, a styrene-butadiene rubber, an acrylated styrene-butadiene rubber, an epoxy resin, nylon, and the like.

A content of the binder may be about 0.1 wt % to about 5 wt %, based on a total weight of the positive electrode active material layer in the positive electrode active material layer.

### Conductive Material

The positive electrode active material layer may further include a conductive material. The conductive material may impart conductivity to the electrode, and a suitable material that does not cause chemical change and conducts electrons can be used in the battery. Examples thereof may include a carbon material such as natural graphite, artificial graphite, carbon black, acetylene black, ketjen black, a carbon fiber, a carbon nanofiber, a carbon nanotube, and the like; a metal material including copper, nickel, aluminum, silver, etc. in a form of a metal powder or a metal fiber; a conductive polymer such as a polyphenylene derivative; or a mixture thereof.

A content of the conductive material in the positive electrode active material layer may be about 0 wt % to about 3 wt %, about 0.01 wt % to about 2 wt %, or about 0.1 wt % to about 1 wt %, based on a total weight of the positive electrode active material layer.

The solid electrolyte may be included in an amount of about 0.1 wt % to about 35 wt %, e.g., about 1 wt % to about 35 wt %, about 5 wt % to about 30 wt %, about 8 wt % to about 25 wt %, or about 10 wt % to about 20 wt %, based on a total weight of the positive electrode active material layer.

In an implementation, the positive electrode active material may be included in an amount of about 5 wt % to about 99 wt %, the solid electrolyte may be included in an amount of about 1 wt % to about 35 wt %, e.g., the positive electrode active material may be included in an amount of about 80 wt % to about 90 wt %, and the solid electrolyte may be included in an amount of about 10 wt % to about 20 wt % based on a total weight of the positive electrode active material and solid electrolyte in the positive electrode active material layer. Including the solid electrolyte in the positive electrode within these amounts may help ensure that the efficiency and cycle-life characteristics of the all-solid-state battery can be improved without reducing the capacity.

The positive electrode current collector may include an aluminum foil.

### Negative Electrode

A negative electrode for an all-solid-state rechargeable battery may include a current collector and a negative electrode active material layer on the current collector. In an implementation, the negative electrode active material layer may include a negative electrode active material, may further include binder or conductive material, may optionally include the aforementioned solid electrolyte, and optionally the additive represented by Chemical Formula 1.

The negative electrode active material may include a material that reversibly intercalates/deintercalates lithium ions, a lithium metal, a lithium metal alloy, a material capable of doping/dedoping lithium, or transition metal oxide.

The material that reversibly intercalates/deintercalates lithium ions may include, e.g., crystalline carbon, amorphous carbon, or a combination thereof as a carbon negative electrode active material. The crystalline carbon may be irregular, sheet-shaped, flake-shaped, sphere-shaped, or fiber-shaped natural graphite or artificial graphite. The amorphous carbon may be a soft carbon, a hard carbon, a mesophase pitch carbonization product, calcined coke, and the like.

The lithium metal alloy may include an alloy of lithium and a metal, e.g., Na, K, Rb, Cs, Fr, Be, Mg, Ca, Sr, Si, Sb, Pb, In, Zn, Ba, Ra, Ge, Al, or Sn.

The material capable of doping/dedoping lithium may be a Si negative electrode active material or a Sn negative electrode active material. The Si negative electrode active material may include silicon, a silicon-carbon composite, SiOx (0<x<2), a Si-Q alloy (wherein Q is an alkali metal, an alkaline-earth metal, a Group 13 element, a Group 14 element, a Group 15 element, a Group 16 element, a transition metal, a rare earth element, or a combination thereof, but not Si) and the Sn negative electrode active material may include Sn, SnO₂, a Sn—R alloy (wherein R is an alkali metal, an alkaline-earth metal, a Group 13 element, a Group 14 element, a Group 15 element, a Group 16 element, a transition metal, a rare earth element, or a combination thereof, but not Sn). At least one of these materials may be mixed with SiO₂. The elements Q and R may be Mg, Ca, Sr, Ba, Ra, Sc, Y, Ti, Zr, Hf, Rf, V, Nb, Ta, Db, Cr, Mo, W, Sg, Tc, Re, Bh, Fe, Pb, Ru, Os, Hs, Rh, Ir, Pd, Pt, Cu, Ag, Au, Zn, Cd, B, Al, Ga, Sn, In, TI, Ge, P, As, Sb, Bi, S, Se, Te, Po, or a combination thereof.

In an implementation, the negative electrode active material may include silicon-carbon composite particles. An average particle diameter (D₅₀) of the silicon-carbon composite particles may be, e.g., about 0.5 μm to about 20 μm. The average particle diameter (D₅₀) is measured with a particle size analyzer and means a diameter of particles with a cumulative volume of 50 volume % in the particle size distribution. Silicon may be included in an amount of about 10 wt % to about 60 wt % and carbon may be included in an amount of about 40 wt % to about 90 wt %, based on a total weight of the silicon-carbon composite particles. In an implementation, the silicon-carbon composite particles may include a core including silicon particles, and a carbon coating layer on the surface of the core. An average particle diameter (D₅₀) of the silicon particles may be about 10 nm to about 1 μm or about 10 nm to about 200 nm in the core. The silicon particles may exist as silicon alone (e.g., non-compounded silicon), in the form of a silicon alloy, or in an oxidized form. The oxidized form of silicon may be represented by SiOx (0<x<2). In an implementation, a thickness of the carbon coating layer may be about 5 nm to about 100 nm.

In an implementation, the silicon-carbon composite particles may include a core including silicon particles and crystalline carbon, and a carbon coating layer on the surface of the core and including amorphous carbon. In an implementation, in the silicon-carbon composite particles, amorphous carbon may not exist in the core but only in the carbon coating layer. The crystalline carbon may be artificial graphite, natural graphite, or a combination thereof, and the amorphous carbon may be formed from coal pitch, mesophase pitch, petroleum pitch, coal oil, heavy petroleum oil, or a polymer resin (phenolic resin, furan resin, polyimide, etc.). In an implementation, a content of the crystalline carbon may be about 10 wt % to about 70 wt % and a content of the amorphous carbon may be about 20 wt % to about 40 wt %, based on a total weight of the silicon-carbon composite particles.

In the silicon-carbon composite particle, the core may include a void in the center. A radius of the void may be about 30 length % to about 50 length % of the radius of the silicon-carbon composite particle.

The aforementioned silicon-carbon composite particles may help effectively suppress volume expansion, structural collapse, or particle crushing due to charging and discharging, prevent disconnection of conductive paths, achieve high capacity and high efficiency, and may be advantageous to use under a high-voltage or high-speed charging conditions.

The Si negative electrode active material or Sn negative electrode active material may be used by mixing with a carbon negative electrode active material. A mixture of Si negative electrode active material or Sn negative electrode active material and carbon negative electrode active material may be mixed at a mixing ratio of about 1:99 to about 90:10 by weight.

A content of the negative electrode active material in the negative electrode active material layer may be about 95 wt % to about 99 wt %, based on a total weight of the negative electrode active material layer.

In an implementation, the negative electrode active material layer may further include the binder and optionally may further include the conductive material. A content of the binder in the negative electrode active material layer may be about 1 wt % to about 5 wt %, based on a total weight of the negative electrode active material layer. In an implementation, a conductive material may be further included, and the negative electrode active material layer may include about 90 wt % to about 98 wt % of the negative electrode active material, about 1 wt % to about 5 wt % of the binder, and about 1 wt % to about 5 wt % of the conductive material.

The binder may well adhere the negative electrode active material particles to each other and also adhere the negative electrode active material to the current collector. The binder may be a water-insoluble binder, a water-soluble binder, or a combination thereof.

The water-insoluble binder may be polyvinyl chloride, carboxylated polyvinyl chloride, polyvinyl fluoride, an ethylene oxide-containing polymer, an ethylene propylene copolymer, polystyrene, polyvinylpyrrolidone, polyurethane, polytetrafluoro ethylene, polyvinylidene fluoride, polyethylene, polypropylene, polyamideimide, polyimide, or a combination thereof.

The water-soluble binder may include a rubber binder or a polymer resin binder. The rubber binder may be a styrene-butadiene rubber, an acrylated styrene-butadiene rubber, an acrylonitrile-butadiene rubber, an acrylic rubber, a butyl rubber, a fluororubber, or a combination thereof. The polymer resin binder may be polyethylene oxide, polyvinylpyrrolidone, polyepichlorohydrin, polyphosphazene, polyacrylonitrile, an ethylene propylene diene copolymer, polyvinylpyridine, chlorosulfonated polyethylene, latex, a polyester resin, an acrylic resin, a phenol resin, an epoxy resin, polyvinyl alcohol, or a combination thereof.

In an implementation, a water-soluble binder may be used as the negative electrode binder, and a cellulose compound capable of imparting viscosity as a type of thickener may be further included. The cellulose compound may include carboxymethyl cellulose, hydroxypropylmethyl cellulose, methyl cellulose, or an alkali metal salt thereof. The alkali metal may be Na, K, or Li. The amount of the thickener used may be about 0.1 parts by weight to about 3 parts by weight, based on 100 parts by weight of the negative electrode active material.

The conductive material may impart conductivity to the electrode, and a suitable material that does not cause chemical change and conducts electrons can be used in the battery. Examples of the conductive material may include a carbon material such as natural graphite, artificial graphite, carbon black, acetylene black, ketjen black, a carbon fiber, a carbon nanofiber, a carbon nanotube, and the like; a metal material of a metal powder or a metal fiber including copper, nickel, aluminum, silver, and the like; a conductive polymer such as a polyphenylene derivative; or a mixture thereof.

The negative current collector may include a copper foil, a nickel foil, a stainless steel foil, a titanium foil, a nickel foam, a copper foam, a polymer substrate coated with a conductive metal, or a combination thereof.

In an implementation, the negative electrode for an all-solid-state rechargeable battery may be a precipitation-type negative electrode. The precipitation-type negative electrode does not include a negative electrode active material during battery assembly, but may refer to a negative electrode in which lithium metal, etc. is precipitated or electrodeposited on the negative electrode during battery charging, thereby serving as a negative electrode active material.

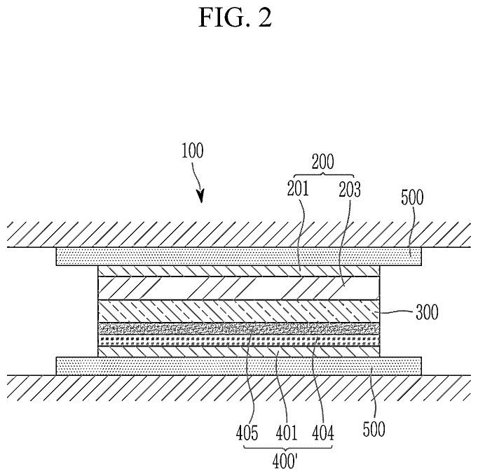

FIG. 2 is a schematic cross-sectional view of an all-solid-state rechargeable battery including a precipitation-type negative electrode. Referring to FIG. 2, the precipitation-type negative electrode 400′ may include a negative electrode current collector 401 and a negative electrode coating layer 405 on the negative electrode current collector 401. In an all-solid-state rechargeable battery having such a precipitation-type negative electrode 400′, initial charging may begin in the absence of negative electrode active material, and during charging, high-density lithium metal is precipitated or electrodeposited between the negative electrode current collector 401 and the negative electrode coating layer 405 or on the negative electrode coating layer 405 to form a lithium metal layer 404, which may serve as a negative electrode active material. Accordingly, in an all-solid-state rechargeable battery that has been charged at least once, the precipitation-type negative electrode 400′ may include, e.g., a negative electrode current collector 401, a lithium metal layer 404 on the negative electrode current collector 401, and a negative electrode coating layer 405 on the lithium metal layer 404. The lithium metal layer 404 may be referred to as a layer in which lithium metal, etc. is precipitated during the charging process of the battery, and may be referred to as a metal layer, lithium layer, lithium electrodeposition layer, or negative electrode active material layer.

In this case, the aforementioned area or the first solid electrolyte layer may be referred to as a surface in contact with the negative electrode coating layer 405.

The negative electrode coating layer 405 may also be referred to as a lithium electrodeposition inducing layer or a negative electrode catalyst layer, and may include a metal, a carbon material, or a combination thereof that acts as a catalyst.

The metal may be a lithiophilic metal and may include, e.g., gold, platinum, palladium, silicon, silver, aluminum, bismuth, tin, zinc, or a combination thereof, and may be composed of one of these or various types of alloys. In an implementation, the metal may be present in particle form, and an average particle diameter (D₅₀) thereof may be less than or equal to about 4 μm, e.g., about 10 nm to about 4 μm.

The carbon material may be, e.g., crystalline carbon, amorphous carbon, or a combination thereof. The crystalline carbon may be for example natural graphite, artificial graphite, mesophase carbon microbeads, or a combination thereof. The amorphous carbon may be, e.g., carbon black, activated carbon, acetylene black, denka black, ketjen black, or a combination thereof.

In an implementation, the negative electrode coating layer 405 may include the metal and the carbon material, and the metal and the carbon material may be, e.g., mixed in a weight ratio of about 1:10 to about 2:1. Herein, the precipitation of the lithium metal may be effectively promoted and improve characteristics of the all-solid-state battery. The negative electrode coating layer 405 may include, e.g., a carbon material on which a catalyst metal is supported or a mixture of metal particles and carbon material particles.

The negative electrode coating layer 405 may include, e.g., the lithiophilic metal and amorphous carbon, and in this case, the deposition of lithium metal may be effectively promoted. In an implementation, the negative electrode coating layer 405 may include a composite in which a lithiophilic metal is supported on amorphous carbon.

The negative electrode coating layer 405 may further include a binder, and the binder may be, e.g., a conductive binder. In an implementation, the negative electrode coating layer 405 may further include an additive, e.g., a filler, a dispersant, an ion conductive agent, or the like.

A thickness of the negative electrode coating layer 405 may be, e.g., about 100 nm to about 20 μm, about 500 nm to about 10 μm, or about 1 μm m to about 5 μm.

The precipitation-type negative electrode 400′ may further include a thin film, e.g., on the surface of the current collector or between the current collector and the negative electrode catalyst layer. The thin film may include an element capable of forming an alloy with lithium. The element capable of forming an alloy with lithium may be, e.g., gold, silver, zinc, tin, indium, silicon, aluminum, bismuth, or the like, which may be used alone or an alloy of more than one. The thin film may further planarize a precipitation shape of the lithium metal layer 404 and may greatly improve characteristics of the all-solid-state rechargeable battery. The thin film may be formed, e.g., using a vacuum deposition method, a sputtering method, a plating method, and the like. The thin film may have, e.g., a thickness of about 1 nm to about 500 nm.

The lithium metal layer 404 may include lithium metal or lithium alloy. In an implementation, the lithium alloy may be Li—Al alloy, Li—Sn alloy, Li—In alloy, Li—Ag alloy, Li—Au alloy, Li—Zn alloy, Li—Ge alloy, or Li—Si alloy.

A thickness of the lithium metal layer 404 may be about 1 μm to about 500 μm, about 1 μm to about 200 μm, about 1 μm to about 100 μm, or about 1 μm to about 50 μm. If the thickness of the lithium metal layer 404 were to be too thin, it could be difficult to perform the role of a lithium storage. If it were to be too thick, the battery volume could increase and performance could deteriorate.

In an implementation, such a precipitation-type negative electrode may be used, and the negative electrode coating layer 405 may serve to protect the lithium metal layer 404 and suppress the precipitation growth of lithium dendrite. Accordingly, short circuit and capacity degradation of the all-solid-state battery may be suppressed and cycle-life characteristics can be improved.

### All-Solid-State Rechargeable Battery

Some embodiments provide an all-solid-state rechargeable battery including a positive electrode, a negative electrode, and a solid electrolyte membrane between the positive electrode and the negative electrode. Herein, at least one of the positive electrode, the negative electrode, and the solid electrolyte membrane may include the aforementioned additive represented by Chemical Formula 1.

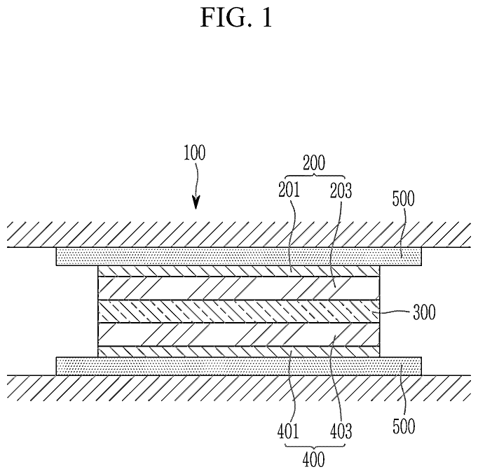

FIG. 1 is a cross-sectional view of an all-solid-state rechargeable battery according to some embodiments. Referring to FIG. 1, the all-solid-state battery 100 may have a structure that an electrode assembly, in which a negative electrode 400 including a negative electrode current collector 401 and a negative electrode active material layer 403, a solid electrolyte layer 300, and a positive electrode 200 including a positive electrode active material layer 203 and a positive electrode current collector 201 are stacked, is housed in a battery case. The all-solid-state battery 100 may further include an elastic layer 500 on the outside of at least either one of the positive electrode 200 and the negative electrode 400. In an implementation, as illustrated in FIG. 1, one electrode assembly may include the negative electrode 400, the solid electrolyte layer 300, and the positive electrode 200, or an all-solid-state rechargeable battery can also be manufactured by stacking two or more electrode assemblies.

An all-solid-state rechargeable battery may be a unit cell with a structure of positive electrode/solid electrolyte layer/negative electrode, a bicell with a structure of negative electrode/solid electrolyte layer/positive electrode/solid electrolyte layer/negative electrode, or a stacked battery in which the structure of the unit cell is repeated.

The shape of the all-solid-state rechargeable battery may be, e.g., coin-shaped, button-shaped, sheet-shaped, stacked-shaped, cylindrical, flat, etc. In an implementation, the all-solid-state rechargeable battery may be applied to a large-sized battery used in an electric vehicle or the like. In an implementation, the all-solid-state rechargeable battery may also be used in hybrid vehicles such as plug-in hybrid electric vehicles (PHEV). In an implementation, it may be used in a field requiring a large amount of power storage, and may be used, for example, in an electric bicycle or a power tool. In an implementation, the all-solid-state rechargeable battery may be used in various fields such as portable electronic devices.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Evaluation Example 1

In order to test a crosslinking polymerization reaction, Reference Example 1 was prepared by adding a compound represented by Chemical Formula 1-1 as an additive to an isobutyl isobutyrate (IBIB) solvent.

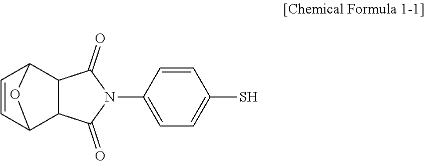 [SMILES: O=C1C2C3C=CC(O3)C2C(=O)N1c1ccc(S)cc1]

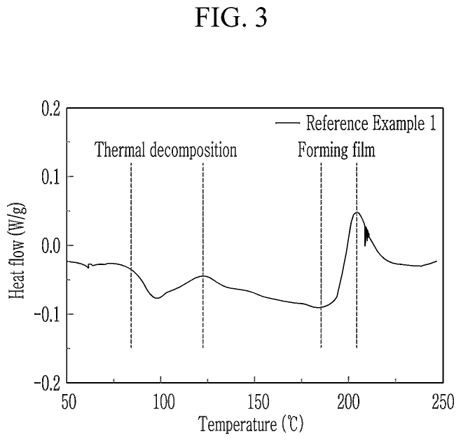

A differential scanning calorimetry (DSC) analysis was performed on Reference Example 1, and the result is shown in FIG. 3. Referring to FIG. 3, in Reference Example 1, a thermal decomposition reaction began at about 80° C. to about 125° C., and a film was formed at about 180° C. to about 205° C. In other words, in Reference Example 1 a polymerization reaction occurred at about 110° C. to about 180° C. and thus formed the film under the high temperature condition.

### Comparative Example 1

A composition for a solid electrolyte membrane was prepared by adding 2 wt % of an acryl copolymer binder (SX-A334, Zeon Corp.) and 98 wt % of a solid electrolyte (Li₆PS₅Cl, D₅₀-3.5 μm) to an IBIB solvent and then, mixing them. The composition was coated on a release PET film with a blade coater and then, pre-dried at about 50° C. and then, dried at about 70° C. under a vacuum condition to form an about 200 μm-thick solid electrolyte membrane.

### Example 1

A solid electrolyte membrane was formed substantially in the same manner as in Comparative Example 1 except that 2 wt % of a binder, 5 wt % of the additive (Chemical Formula 1-1) used in Reference Example 1, and 93 wt % of a solid electrolyte were used to form the solid electrolyte membrane.

### Evaluation Example 2

The solid electrolyte membranes according to Comparative Example 1 and Example 1 were respectively heated at 130° C. for 15 minutes to measure ionic conductivity changes before and after the heating, and the results are shown in Table 1. The ionic conductivity was measured through an electrochemical impedance spectroscopy (EIS) analysis, and the EIS analysis was performed at an amplitude of about 10 mV and a frequency of 0.001 Hz to 0.1 Hz under an air atmosphere at 25° C.

TABLE 1

Lithium ionic conductivity (mS/cm)

| | before heating | after heating |
|---|---|---|
| Comparative Example 1 | 0.53 | 0.51 |
| Example 1 | 0.45 | — |

(In Table 1, ‘-’ means that the ionic conductivity was so low that it was not measured)

Referring to Table 1, in Example 1, a film was formed in the solid electrolyte membrane at 130° C., leading to a shutdown, which confirms that the ionic conductivity was successfully reduced. On the contrary, in Comparative Example 1, a film was not formed after heating at 130° C., failing to lead to a shutdown, which confirms that the ionic conductivity was almost unchanged.

One or more embodiments may provide a solid electrolyte membrane and electrode that can block ion currents and induce battery shutdown by forming a film at high temperature during an abnormal reaction in an all-solid-state rechargeable battery, and an all-solid-state rechargeable battery with improved safety and reliability.

The solid electrolyte membrane and electrode according to some embodiments may form a film when the temperature rises due to an abnormal reaction of the battery, blocking ion current and inducing shutdown of the battery, thereby improving safety and reliability of the all-solid-state rechargeable battery.

Example embodiments have been disclosed herein, and although specific terms are employed, they are used and are to be interpreted in a generic and descriptive sense only and not for purposes of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A solid electrolyte membrane, comprising:
a sulfide solid electrolyte; and
an additive,
wherein:
the additive includes a compound represented by Chemical Formula 1, 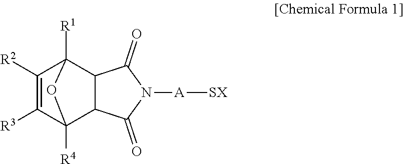
in Chemical Formula 1,
A is a substituted or unsubstituted C1 to C20 alkylene group, a substituted or unsubstituted C3 to C20 cycloalkylene group, a substituted or unsubstituted C6 to C20 arylene group, a substituted or unsubstituted C6 to C20 heteroarylene group, or a combination thereof,
S is sulfur,
X is hydrogen, Li, Na, K, Rb, Cs, Fr, Mg, Ca, Ba, Cu, Zn, Ag, In, Sb, Co, Fe, Mn, or Pd, and
R¹ to R⁴ are each independently hydrogen, a substituted or unsubstituted C1 to C20 alkyl group, a substituted or unsubstituted C2 to C20 alkenyl group, a substituted or unsubstituted C2 to C20 alkynyl group, a substituted or unsubstituted C6 to C20 aryl group, a substituted or unsubstituted C3 to C20 cycloalkyl group, a substituted or unsubstituted C3 to C20 cycloalkenyl group, a substituted or unsubstituted C3 to C20 cycloalkynyl group, a substituted or unsubstituted C2 to C20 heterocycloalkyl group, a substituted or unsubstituted C2 to C20 heterocycloalkenyl group, a substituted or unsubstituted C2 to C20 heterocycloalkynyl group, a substituted or unsubstituted C3 to C20 heteroaryl group, a substituted or unsubstituted C1 to C20 alkoxy group, an ester group, an ether group, a carboxyl group or a salt thereof, a cyano group, a carbonyl group, an imino group, a halogen, N, O, S, P, Si, B, or a combination thereof.

2. The solid electrolyte membrane as claimed in claim 1, wherein, in Chemical Formula 1, A is a substituted or unsubstituted C6 to C20 arylene group, a substituted or unsubstituted C6 to C20 heteroarylene group, or a combination thereof.

3. The solid electrolyte membrane as claimed in claim 1, wherein, in Chemical Formula 1, X is hydrogen, Li, Na, or K.

4. The solid electrolyte membrane as claimed in claim 1, wherein, in Chemical Formula 1, R¹ to R⁴ are each independently hydrogen, a substituted or unsubstituted C1 to C20 alkyl group, a substituted or unsubstituted C6 to C20 aryl group, a halogen, N, O, S, P, Si, B, or a combination thereof.

5. The solid electrolyte membrane as claimed in claim 1, wherein, in Chemical Formula 1,
A is a substituted or unsubstituted C6 to C20 arylene group,
X is hydrogen, Li, or Na, and
R¹ to R⁴ are each independently hydrogen or a substituted or unsubstituted C1 to C20 alkyl group.

6. The solid electrolyte membrane as claimed in claim 1, wherein the additive forms a film in a temperature range of about 110° C. to about 180° C.

7. The solid electrolyte membrane as claimed in claim 1, wherein the additive is included in an amount of about 1 wt % to about 10 wt %, based on a total weight of the solid electrolyte membrane.

8. The solid electrolyte membrane as claimed in claim 1, wherein the additive is included in an amount of about 1 wt % to about 5 wt %, based on a total weight of the solid electrolyte membrane.

9. The solid electrolyte membrane as claimed in claim 1, wherein:
the solid electrolyte membrane includes a film including a compound represented by Chemical Formula 4: 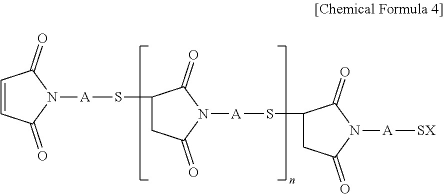
in Chemical Formula 4,
A is a substituted or unsubstituted C1 to C20 alkylene group, a substituted or unsubstituted C3 to C20 cycloalkylene group, a substituted or unsubstituted C6 to C20 arylene group, a substituted or unsubstituted C6 to C20 heteroarylene group, or a combination thereof,
S is sulfur,
X is hydrogen, Li, Na, K, Rb, Cs, Fr, Mg, Ca, Ba, Cu, Zn, Ag, In, Sb, Co, Fe, Mn, or Pd, and
n is an integer of 2 to 500.

10. The solid electrolyte membrane as claimed in claim 9, wherein, in Chemical Formula 4, A is a substituted or unsubstituted C6 to C20 arylene group, a substituted or unsubstituted C6 to C20 heteroarylene group, or a combination thereof.

11. The solid electrolyte membrane as claimed in claim 9, wherein, in Chemical Formula 4, X is hydrogen, Li, Na, or K.

12. The solid electrolyte membrane as claimed in claim 9, wherein, in Chemical Formula 4,
A is a substituted or unsubstituted C6 to C20 arylene group, and
X is hydrogen, Li, or Na.

13. The solid electrolyte membrane as claimed in claim 1, wherein:
the sulfide solid electrolyte is in the form of particles, and
an average particle diameter (D₅₀) of the particles is about 0.1 μm to about 5.0 μm.

14. The solid electrolyte membrane as claimed in claim 1, wherein:
the sulfide solid electrolyte includes an argyrodite-type sulfide represented by Chemical Formula 5:
(LiₐM¹_{b}M²_{c})(P_{d}M³ₑ)(S_{f}M⁴_{g})Yₕ [Chemical Formula 5]
in Chemical Formula 5,
4<a≤8, 0≤b≤0.5, 0≤c≤0.5, 0<d<4, 0≤e<1, 1.5≤n≤5, 3≤f≤12, 0≤g<2, and 0≤h≤2,
M¹ is Mg, Cu, Ag, or a combination thereof,
M² is Na, K, or a combination thereof,
M³ is Sn, Zn, Si, Sb, Ge, or a combination thereof,
M⁴ is O, SOₙ, or a combination thereof, and
Y is F, Cl, Br, I, or a combination thereof.

15. An electrode for an all-solid-state rechargeable battery, the electrode comprising:
an electrode active material,
sulfide solid electrolyte particles, and
an additive represented by Chemical Formula 1: 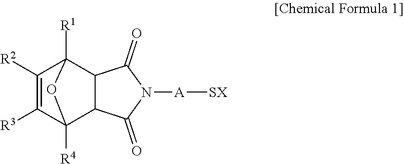
wherein, in Chemical Formula 1,
A is a substituted or unsubstituted C1 to C20 alkylene group, a substituted or unsubstituted C3 to C20 cycloalkylene group, a substituted or unsubstituted C6 to C20 arylene group, a substituted or unsubstituted C6 to C20 heteroarylene group, or a combination thereof,
S is sulfur,
X is hydrogen, Li, Na, K, Rb, Cs, Fr, Mg, Ca, Ba, Cu, Zn, Ag, In, Sb, Co, Fe, Mn, or Pd, and
R¹ to R⁴ are each independently hydrogen, a substituted or unsubstituted C1 to C20 alkyl group, a substituted or unsubstituted C2 to C20 alkenyl group, a substituted or unsubstituted C2 to C20 alkynyl group, a substituted or unsubstituted C6 to C20 aryl group, a substituted or unsubstituted C3 to C20 cycloalkyl group, a substituted or unsubstituted C3 to C20 cycloalkenyl group, a substituted or unsubstituted C3 to C20 cycloalkynyl group, a substituted or unsubstituted C2 to C20 heterocycloalkyl group, a substituted or unsubstituted C2 to C20 heterocycloalkenyl group, a substituted or unsubstituted C2 to C20 heterocycloalkynyl group, a substituted or unsubstituted C3 to C20 heteroaryl group, a substituted or unsubstituted C1 to C20 alkoxy group, an ester group, an ether group, a carboxyl group or a salt thereof, a cyano group, a carbonyl group, an imino group, a halogen, N, O, S, P, Si, B, or a combination thereof.

16. The electrode for an all-solid-state rechargeable battery as claimed in claim 15, wherein the additive is included in an amount of about 1 wt % to about 10 wt %, based on a total weight of the electrode.

17. An all-solid-state rechargeable battery, comprising:
a positive electrode,
a negative electrode, and
the solid electrolyte membrane as claimed in claim 1 between the positive electrode and the negative electrode.

18. The all-solid-state rechargeable battery as claimed in claim 17, wherein the negative electrode includes:
a current collector,
a negative electrode coating layer on the current collector and including a lithiophilic metal, a carbon material, or a combination thereof, and
a lithium metal layer between the current collector and the negative electrode coating layer, the lithium metal layer being formed by charging.
